(19) United States
(12) Patent Application Publication
(10) Pub. No.: US 2013/0225428 A1
(43) Pub. Date: Aug. 29, 2013

(54) LIVER DISEASE MARKERS

(71) Applicant: Institute For Systems Biology, (US)

(72) Inventors: Shizhen QIN, Mill Creek, WA (US); Leroy Hood, Seattle, WA (US)

(73) Assignee: Institute for Systems Biology, Seattle, WA (US)

(21) Appl. No.: 13/691,170

(22) Filed: Nov. 30, 2012

Related U.S. Application Data
(60) Provisional application No. 61/565,383, filed on Nov. 30, 2011.

(51) Int. Cl. C12Q 1/68; G01N 33/68

(52) U.S. Cl. CPC C12Q 1/6876; G01N 33/6893
USPC 506/9; 506/18; 506/16

(57) ABSTRACT

New markers for diseased liver conditions have been identified. A combination of protein C (PROC) and retinol binding protein 4 (RBP4) in blood are biomarkers to distinguish patients at different stages of hepatic fibrosis due, for example, to chronic infection with hepatitis C virus (HCV). Also, alpha-1-B glycoprotein (A1BG), complement factor H (CFH) and insulin-like growth factor binding protein acid labile subunit (IGFALS) distinguish subjects with chronic or acute liver conditions such as hepatitis infection or liver fibrosis from healthy controls.

### CROSS-REFERENCE TO RELATED APPLICATION

This application claims priority under 35 U.S.C. §119(e) to provisional U.S. application Ser. No. 61/565,383 filed 30 Nov. 2011. The contents of this document are incorporated herein by reference in their entirety.

### STATEMENT OF RIGHTS TO INVENTIONS MADE UNDER FEDERALLY SPONSORED RESEARCH

This work was supported in part by grants from the National Institutes of Health. The U.S. government has certain rights in this invention.

### TECHNICAL FIELD

The invention is in the field of diagnostics for liver disease. More particularly it relates to new markers for Hepatitis C infection and for liver fibrosis.

### BACKGROUND ART

Diagnosis of liver conditions due to virus, chemicals, drugs or metabolic disorders has been uncertain for many years. Biopsy is considered the gold standard, but it is not completely reliable in view of the lack of uniformity in distribution of diseased portions of the liver. It is also invasive and has a number of complications and risks. Others have attempted to find markers that would be monitored in serum or blood with varying degrees of success.

Human liver is especially an important target for damage by hepatitis. Liver biopsy is recommended in the management of patients with chronic Hepatitis C (CHC) to provide important information about fibrosis stage and disease prognosis. As an invasive procedure, liver biopsy is frequently accompanied by transient pain and may occasionally be associated with serious complications. The accuracy of liver biopsy in staging liver disease is limited by the size and quality of the samples and sampling error.

In recent years, intensive research in the field of noninvasive tests of liver fibrosis has yielded a few laboratory markers, which enabled the assessment of some aspects of the severity of Hepatitis C virus (HCV)-induced liver disease. For example, the FibroTest™, combines six serum markers (Alpha-2-macroglobulin, Haptoglobin, Apolipoprotein A1, Gamma-glutamyl transpeptidase, Alanine transaminase and total bilirubin) with the age and gender of the patient to generate a score that correlates with stage of fibrosis in patients with a variety of liver diseases. Platelet counts, AST/ALT ratio, and AST-platelet ratio index (APRI) have been reported as predictors of degree of fibrosis in CHC patients. In the Hepatitis C Antiviral Long-term Treatment against Cirrhosis (HALT-C) Trial, a model based on a combination of standard laboratory tests comprising platelet count, AST/ALT ratio, and INR (international normalized ratio of prothrombin time) predicted histological cirrhosis with high accuracy in 50% of patients with CHC.

Two proteins described below, PROC and RBP4 have been related to liver diseases. The plasma PROC level of patients with liver damage due to chronic alcohol consumption was disclosed as decreased and correlated with clinical performance of the patients (Kloczko, J., et al., Haemostasis (1992) 22:340-344). Serum concentrations of RBP4 were reported significantly higher in obese children with non-alcoholic fatty liver disease (NAFLD) compared to controls and proposed RBP4 as a serum marker of intrahepatic lipid content in obese children (Romanowska, A., et al., Acta Biochim Pol (2011) 58:35-38). A contradictory report showed serum RBP4 levels were not different between the steatosis group and controls as well as between subgroups with high and normal ALT, indicating that serum RBP4 may not be a predictive factor in NAFLD (Cengiz, C., et al., Eur J Gastroenterol Hepatol (2010) 22:813-819).

All documents cited herein are incorporated by reference in their entirety.

Molecular signatures specific for the liver and/or specific for particular causes of liver damage would be very useful for experimental, clinical, and epidemiology studies of liver diseases. There remains a need for noninvasive techniques to provide information with respect to disease status and prognosis.

### DISCLOSURE OF THE INVENTION

Through development of targeted proteomic assays utilizing Selected Reaction Monitoring (SRM) incorporating heavy-isotope doping of labeled matched peptides (Picotti, P., et al., Cell (2009) 138:795-806 and Picotti, P., et al., Nat Methods (2010) 7:43-46), and use of the Human SRMAtlas with optimized transitions associated with typically six different peptides for nearly all of the 20,300 human protein-coding genes, accurate quantitation of target proteins can be achieved for most human proteins present at levels that can be detected by targeted mass spectrometry. SRM proteomics have been employed to identify many liver-specific proteins and to characterize the critical progression of fibrosis of the liver to cirrhosis in CHC patients.

The invention thus resides in the identification of new markers that are capable of identifying individuals who are infected with hepatitis or exhibit chronic liver disease generally. The markers of the invention may also be used to establish the status of liver fibrosis in relation to a commonly recognized scale of severity, the Ishak score which ranks severity of fibrosis based on histological criteria from a minimum of 0 to a maximum disease state of 6.

Thus, in one aspect, the invention is directed to a method to distinguish a subject infected with hepatitis virus from a non-infected subject or more generally, a subject with an abnormal liver condition from a normal subject which method comprises assessing the level of alpha-1-B glycoprotein (A1BG) or complement Factor H (CFH) or insulin-like growth factor binding protein acid labeled subunit (IGFALS) or combinations thereof in the blood or fraction thereof of said subject whereby a statistically significant increased level of A1BG or a statistically significant decreased level of CFH or IGFALS or combinations thereof as compared to normal individuals identifies said subject as infected with hepatitis or afflicted with abnormal liver condition generally. Particularly important is the ability to detect infection with Hepatitis C.

While the example below develops the method and verifies it with respect to a cohort of patients infected with Hepatitis C which is a chronic condition, the markers of the invention listed above will also identify patients with acute liver conditions that are abnormal.

In another aspect, the same criteria as mentioned in the previous paragraph may be used to distinguish subjects with liver fibrosis or other abnormal liver condition from those not affected with this condition.

The invention is also directed to a panel of reagents for detecting the foregoing markers, A1BG, CFH, and IGFALS. The panel may contain reagents for detection of only one or two or three of these markers. Typical reagents would include, for example, antibodies or portions thereof, aptamers, and the like.

In still another aspect, the invention is directed to a method to determine severity of liver fibrosis in a subject exhibiting liver fibrosis which method comprises determining the level of protein C (PROC) and/or retinal binding protein 4 (RBP4) in the blood or a fraction thereof of a subject wherein statistically significant decreased levels of one or both of said PROC and RBP4 as compared to normal individuals correlates positively with the severity of fibrosis of the liver in said subject. The fibrosis may be due to hepatitis infection or as a result of other causes, such as infections in general, toxic substances, genetic mutations or cancer. In addition, PROC and RBP4 levels as described in the previous paragraph may be added to the panel of markers to distinguish a subject having an abnormal liver condition from a subject who does not in addition to the A1BG and/or CFH and/or IGFALS described above.

Reagents reactive with RBP4 and/or PROC are also included in the invention. These reagents may also be added to the panel for detection of A1BG, CFH and/or IGFLAS.

### BRIEF DESCRIPTION OF THE DRAWINGS

FIG. 1A shows variations between duplicate runs of serum protein levels of IGFALS measured by peptide LHSLHLEGSCLGR were consistent with a Pearson correlation value >0.9. FIG. 1B shows multiple peptides (or features) derived from the same protein performed consistently in most SRM tests. A1BG serum levels measured by the three features SGLSTGWTQLSK and HQFLLTGDTQGR precursor charge 2 and 3 were in close agreement. Relative protein levels in blood are indicated by light/heavy peptide ratios.

FIG. 2A is a box plot analysis showing significant differences when comparing serum protein levels of A1BG, CFH and IGFALS in patients from Ishak fibrosis 2-6 against controls (C). The combined serum A1BG level of all patients is significantly elevated compared to the controls (P=7.4E-15), while the combined serum CFH and IGFALS levels are significantly decreased in patients versus controls (P=4.5E-08 and 5.7E-12, respectively). FIGS. 2B and 2C show PROC and RBP4 levels in serum classify HCV patients in different stages of fibrosis. Box plot analysis shows strong evidence that the median serum levels of PROC and RBP4 in every Ishak fibrosis group decreased with the progression of the disease.

FIG. 3A shows SRM data of serum A1BG and CFH levels in both patients DJ001705 and DJ001652 (arrows) are of good quality; their relative protein levels are probably truly different from other patients. However, this discrepancy can be resolved by examining simultaneously A1BG and CFH or A1BG and IGFALS levels in these patients. FIG. 3B shows a combination of A1BG, CFH and IGFALS is able to discriminate healthy controls from HCV patients from Ishak scores 2 to 6 with predicted high sensitivity and specificity.

FIG. 4 shows protein markers PROC and RBP4 and platelets distinguish patients with cirrhosis (Ishak 5-6) from patients in other stages with sensitivity of 95% and specificity of 84%. Inclusion of additional proteins in this study did not result in meaningful further improvement.

### MODES OF CARRYING OUT THE INVENTION

The invention provides five new useful markers for liver conditions in individuals that permit simple blood tests for their determination. The diagnostic tests are conveniently done on a fraction of the blood such as serum or plasma. While the test is most useful in detecting disease in humans, it is also applicable to other warm-blooded animals such as livestock, household pets, and service animals such as horses. They may also be used in laboratory animals such as disease models. The availability of such tests, particularly in combination with other markers and/or using a multiplicity of the invention markers permits use of a simple test for liver conditions, optionally in combination with other indications that can be performed at the point of care. The availability of multiplexed assays for proteins using miniaturized devices makes these tests especially important in providing fast assays with high reliability. Thus, in addition specifically to identifying subjects infected with hepatitis or afflicted with other liver fibrotic conditions, these markers may be used in combination with markers for other diseases and conditions to provide a detailed assessment of the physiological state of an individual in a single multiplexed assay.

Classifying stages of liver fibrosis by biopsy has an accuracy of about 80% (Grigorescu, M., et al., J. Gastrointestin Liver Dis (2006) 15:149-159). Other studies have suggested that there can be up to a 33% error in the diagnosis of cirrhosis by biopsy (Afdhal, N. H., et al., Am J Gastroenterol (2004) 99:1160-1174). When the value of biomarkers is validated against biopsy, it is improbable to have a discrimination power that exceeds biopsy. In fact, reported noninvasive methods intended for discriminating hepatic fibrosis rarely have an AUROC (accuracy) exceeding 0.8 to 0.9 (Ray, S, et al., Hepatitis C, Churchill, Livingstone, Elsevier, Philadelphia, Pa. 2009). Thus the results shown in the example below with respect to the invention methods and reagents are unexpected and surprising.

The biomarkers described hereinbelow represent a distinct improvement from the reliability of these prior art methods as demonstrated in the example below. The newly discovered markers, A1BG, CFH and IGFALS, may be used individually or in various combinations in panels to distinguish subjects with chronic liver conditions, such as liver infection or fibrosis, from normal controls. Addition of one or both of the markers PROC and RBP4 to the determination may increase the accuracy of the assay even more. The latter two proteins are particularly useful in determining the progression of liver conditions.

One of the important aspects of the invention relates to establishing the state of progression of liver fibrosis. This is generally ranked on an Ishak group from 0 to 6. Mild fibrosis which is representative of the early stage of cancer has a rating of 1. More severe forms of cancer or more progressed cancers have higher ratings on the Ishak scale. Ishak 2 represents a more advanced stage of cancer, for example.

In addition to being used independently, the protein markers of the invention can be used in conjunction with other indicators. For example, low platelet counts are associated with progression of fibrosis. Used alone, this would be non-determinative since it is well known that there are many other causes other than liver diseases of low blood platelet levels (thrombo-cytopenia) such as leukemia, some types of anemia, immune system malfunction, metabolic disorders, viral infections, toxic chemicals or as a medication side effect. These could lead to a decreased platelet production or increased platelet destruction. Addition of liver-specific proteins to a diagnostic test panel increases the diagnostic relevance of platelet counts.

The markers of the invention have been identified by focusing on proteins known to be relatively specific for liver as compared to other organs as discerned from publicly available databases. Typically, proteins normally present at high levels in the serum or plasma sample are first eliminated from the sample and then the levels of diagnostic proteins assessed. A particularly useful method for detecting low levels of proteins in biological samples is selected reaction monitoring (SRM) targeted proteomics which was used to obtain the initial data. Sophisticated statistical analysis procedures were then employed to demonstrate that the five markers that are the subject of the present invention are reliable indicators of the conditions with which they are associated.

Once the nature of the markers for the conditions indicated has been identified, means for measuring their levels and determining the statistical significance of them as compared to levels in normal subjects are well known in the art. A multiplicity of techniques for measuring levels of protein is well established and includes, for example, various immunological based assays, mass spectra based assays and the like. Further, determination of a statistically significant difference in a test subject as compared to normal subjects employs statistical analysis well understood by practitioners. The values in normal subjects may be obtained from the literature, from comparable healthy individuals, or averages of levels from a multiplicity of individuals. If necessary, the levels in normals may be matched for other, perhaps relevant factors such as age, gender, ethnic background and alike. All of this is well within the expertise of the ordinary artisan.

Protein analytes are good targets for developing antibodies or synthetic capture agents that can be integrated into microfluidic chips (Integrated Blood-Barcode Chip)—devices that have the potential to analyze large numbers of patient samples rapidly (in a few minutes), inexpensively, and in a highly multiplexed format (100s or even 1000s of different assays investigating many different diseases) employing blood from a pinprick. Such microfluidic devices are likely to constitute an important foundation for P4 (Predictive, Preventive, Personalized, and Participatory) Medicine with Point-of-Care Diagnosis.

As noted above, panels of reagents for detection of the markers useful in the methods discussed herein are also included in the invention. Thus, the invention includes panels of reagents for use in each of these methods.

In one embodiment, the reagents are antibodies specifically immunoreactive with the markers to be detected. “Antibodies” includes complete antibodies, fragments of the antibodies that are immunoreactive with the protein to be detected, as well as recombinantly produced forms such as single stranded antibodies. The antibodies may be monoclonal or polyclonal and include antibodies derived from any subject since immunogenicity is not an issue in such assays.

Also included in the invention are individual antibodies as described above for each of the five protein markers useful in the methods as well as aptamers that specifically bind these proteins.

The following example is presented to illustrate but not to limit the invention.

### Example 1

### Determination of Markers

This example is a detailed description of identification of the present markers and of the manner in which they can be used to assess chronic conditions of the liver such as infection, liver fibrosis, and fibrosis progression in an individual.

By employing a liver-specific protein strategy (employing comprehensive transcriptomic databases) and targeted quantitative SRM proteomics technology, we have analyzed 38 liver-specific protein levels in sera of 17 healthy controls and of 38 HCV patients at Ishak fibrosis stages from 2 to 6. Two protein markers, protein C (PROC) and retinol binding protein 4 (RBP4) are present at lower levels in patients than in controls. With Area Under the Curve (AUC) statistical analyses, these two proteins distinguish fibrosis vs. cirrhosis among Chronic Hepatitis C (CHC) patients. Three proteins, A1BG, CFH and IGFALS, distinguish HCV-infected patients from healthy controls, with an individual AUROC score >0.96 for each marker.

Serum samples were obtained from patients who participated in the HALT-C Trial (Ghany, M. G., et al., Hepatology (2011) 54:1527-1537). This trial enrolled patients with CHC who had liver biopsies showing Ishak stages 2-6 (range 0-6) fibrosis at enrollment. Blood samples at enrollment were studied. Patient information at enrollment is listed in Table 1 at the end of this example.

Control sera from normal female and male donors ages 30-50 years were collected at FDA-regulated blood facilities and were non-reactive for HCV antibody (ProMedDx). Pooled plasma from 10 normal donors was obtained from Innovative (Novi, Mich.). Collection and use of control and patient samples were approved by institutional review boards. Samples were stored at −80° C.

Sample Preparation for SRM

To reduce the complexity of samples, the top 14 highly abundant proteins were depleted using an AKTA FPLC system (GE Healthcare, USA) coupled with a Seppro® IgY14 human LC2 depletion column (Sigma-Aldrich, USA). We observed significant sample-to-sample variations with the Seppro® IgY14 spin column. In contrast, the LC system coupled with an IgY14 human LC2 depletion column dramatically improved the reproducibility. All 40 HALT-C and 17 normal serum samples were processed similarly; about 95% of the total protein was depleted. Depleted sera were digested with trypsin and desalted with Oasis™ MCX cartridges (Waters, Milford, Mass.).

Building the Liver-Specific and Liver-Enriched Proteins List

We used a targeted approach focusing on organ-specific proteins to increase the likelihood of identifying protein biomarkers in blood that may reflect pathology of a particular organ. Our list of liver-specific or liver-enriched proteins (liver proteins) was created by mining multi-organ transcriptomic data generated through Massively Parallel Signature Sequencing (MPSS). The MPSS dataset contains transcriptomes of 34 pooled normal (Caucasian) human tissues (Lin, B., et al., PLoS One (2010) 5:e10210). Signatures with their expression levels in liver either 5-fold higher than any other organs or 2-fold greater than the sum of all other organs were selected as liver protein candidates. We also performed organ-specific protein search with Gene Atlas Interface analysis. The databases searched against were 3 datasets from NCBI-GEO (Gene Expression Omnibus) with a total of 180 human tissues from multiple donors (Ge, X., et al., Genomics (2005) 86:127-141; Roth, R. B., et al., Neurogenetics (2006) 7:67-80; and Su, A. I., et al., PNAS USA (2004) 101:6062-6067). We included 21 enzymes and other proteins used in clinical practice or previously reported as liver biomarker candidates. Unfortunately, none of the 21 potential liver markers were successfully detected by SRM except coagulation factor II (F2).

Peptide selection from the Liver Protein List

Two to three peptides were selected for each liver protein based on the sequence of individual liver proteins that were previously detected by mass spectrometry. Peptide selection criteria are as follows (Lange, V., et al., Mol Syst Biol (2008) 4:222): 1) length 8-20 amino acid residues; 2) no chemically unstable residues (single letter notation; M, NG, DG, QG, N-terminal N, and N-terminal Q); 3) LC-compatible; 4) avoid cysteine residue if possible; and 5) sequence specific for the target protein (e.g., proteotypic peptides). Peptides previously identified in PeptideAtlas (Deutsch, E. W., et al., EMBO Rep (2008) 9:429-434) were preferentially chosen.

Mass Spectrometry and HPLC

All SRM analyses were performed on an Agilent 6460A triple quadrupole (QQQ) mass spectrometer with a ChipCube™ nanoelectrospray ionization source coupled with an Agilent 1200 nanoflow HPLC system. Serum samples were eluted over a 60-minute gradient with 0.66% per minute acetonitrile slope in the presence of 0.1% formic acid using a large capacity Agilent HPLC chip (Cat #G4240-62101, 160 mL trap, 150 mm C18 column). Spray voltage was set at 1900 V. The scheduled SRM were performed with 5 min retention time windows and an instrument cycle time of 2000±500 ms. Dwell times varied depending on the number of concurrent transitions; in all cases they were at least 10 ms.

Monitoring Liver-Specific Proteins in Blood by SRM

Crude unpurified peptide standards that correspond to the detected natural counterparts (light peptides) were synthesized with heavy isotopic Lysine (¹³C6¹⁵N2) or Arginine (¹³C6¹⁵N4) at the C-termini (heavy peptides) (Thermo-Fisher Scientific, Germany or Sigma-Aldrich, USA). Collision energies (CE) were determined using the default formula from Agilent (0.036×precursor mass m/z−4.80) and then optimized with 4 additional CE steps (±5V, ±10V). The best 4 transitions were selected. Detected heavy peptides were titrated at 6 concentrations in a normal human serum background to build a titration curve and to determine the proper amount of each peptide standard to spike-in.

SRM Data Analysis

All SRM data were processed using the Skyline Targeted Proteomics Environment (v1.1) (MacLean, B., et al., Bioinformatics (2010) 26:966-968). The setting of 0.055 Th match tolerance m/z was used. The default peak integration and Savitzky-Golay smoothing algorithm were applied. All data were manually inspected to ensure correct peak detection and accurate integration. Peptides with at least 3-fold signal-to-noise ratio were considered detectable. The total peak area and Light/Heavy ratio of each peptide were exported for statistical analysis.

Statistical Analysis

The exported SRM results were analyzed using R scripts generated for this data set. The pre-processing selection included proteins that have no more than 30% missing data. A similar criterion was applied to samples. Missing data were handled using k-nearest neighbor imputation algorithms (k=10) (Troyanskaya, O., et al., Bioinformatics (2001) 17:520-525). Repeated (duplicate) measurements for the same protein-peptide-m/z combination were averaged. Platelet level and gender were included as a clinical predictor of liver damage. Regularization methods based on logistic regression were used to reduce overfitting. LASSO and Elastic Nets penalty (Zou, H., et al., J. Roy. Statist. Soc. Ser. B (2005) 67:301-320) were applied. The choice of the optimal regularization parameter was done using the Area Under the Receiver-Operating-Characteristic (AUROC) curve as a criterion.

In order to obtain an approximately unbiased assessment of the performance of predictive signatures, tenfold cross validation was used to correct for the generally overoptimistic model building and signature optimization bias. An average over cross-validation runs is reported as the final optimal AUC characterizing the predicted performance of the biomarker signature. LASSO penalty was preferred for its ability to drop non-essential biomarkers from the signature by explicitly assigning them zero weights. All other analyses including calculation and graphics were generated by Prism 5 (GraphPad software, La Jolla, Calif., USA).

Results

The Identification of Liver Proteins

Using strategies described above, we identified 109 liver-specific proteins. GeneCards® summarized each gene expression in normal and diseased human tissues by three categories: 1) mRNA expression data from GeneNote and GNF BioGPS, 2) UniGene electronic Northern, and 3) SAGE (Serial Analysis of Gene Expression). In combination with the 21 proteins used in clinic practice or reported as liver biomarker candidates, a list composed of 130 proteins was created.

Proteins Detected by SRM

After suitable peptides and transitions for each liver protein were selected as described above, we used control plasma and serum to determine how many liver proteins can be detected by SRM. From the 89 liver proteins previously observed in tandem MS/MS experiments, we detected 100 peptides derived from 54 proteins in pooled control plasma (Table 2 at the end of this example). However, the HALT-C samples were in the form of serum, which is not ideal for mass spectrometry-based blood protein measurements due to variation in proteolysis derived from the coagulation cascade, resulting in decreased concentrations of proteins compared to plasma. In order to determine how many of our proteins detected in plasma can be detected in serum; we performed the same SRM analyses against pooled healthy human serum samples. Altogether 38 proteins (represented by 65 peptides) were detected in sera with reasonable signal intensity as shown in Table 3 at the end of this example).

Consistency and Accuracy of SRM Data

Duplicate SRM Runs Are Well Correlated

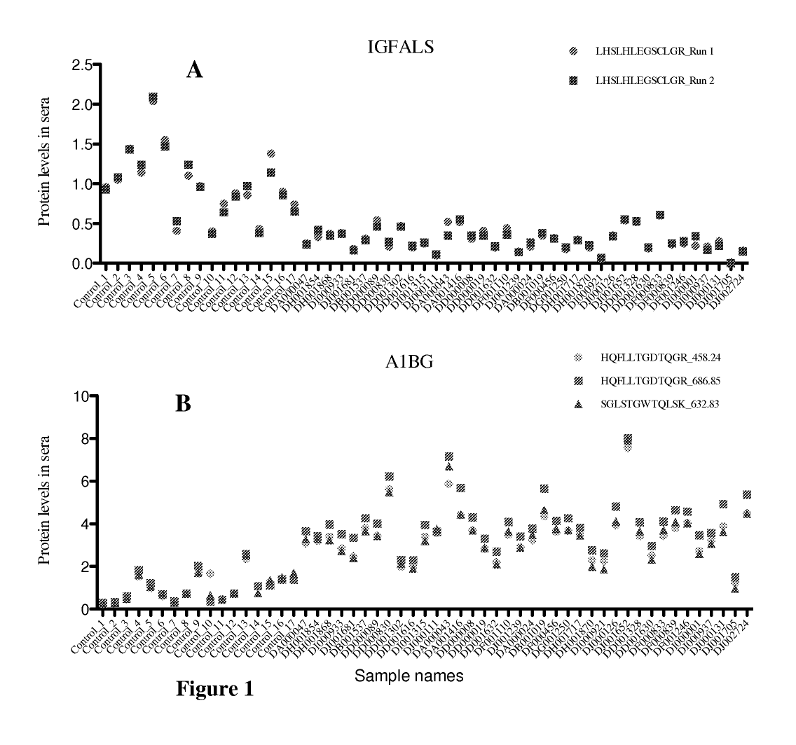

Duplicate runs were performed for each sample; technical variations between the two runs were generally small (FIG. 1A). Pearson tests showed good correlations between runs (≧0.9 in most cases; for example: PROC=0.90, RBP4=0.90, CFH=0.95, A1BG=0.94). Samples from two HALT-C patients, DA000739 and DJ000004 were eliminated from the analysis due to sample degradation.

Protein Levels Measured by Multiple Features Are Consistent

When a protein level is measured by more than one feature (i.e., multiple peptides or same peptide with differently charged precursor ions), close agreement in quantification was observed; an example is the set of three features for the protein of A1BG (FIG. 1B). This observation gave us reasonable confidence that protein levels in samples estimated even from a single peptide can be reliable.

Absolute Protein Levels in Sera Measured by SRM Are Similar to Prior Report

We did not aim to quantify absolute protein concentrations. The crude heavy peptides synthesized by rapid process peptide synthesis were not appropriate for absolute quantitation of protein levels in specimen due to the wide range of purity (−50% to 80%). In addition, the immunodepletion procedure during sample preparation induces additional variation in protein concentrations. Nevertheless, as summarized in Table 4, levels of five informative proteins in depleted control sera measured by SRM are close to concentrations reported in published literatures. The only exception is RBP4; the concentration in our SRM study is 10-fold lower than the studies of Gahne, B., et al. (Hum Genet (1987) 76:111-115) and Polanski, M, et al. (Biomark Insights (2007) 1:1-48) and 100-fold lower than the study of Farrah, et al. (Mol Cell Proteomics (2011) 10:M110 006353).

TABLE 4
Five informative protein concentrations in control sera measured by crude heavy
peptides in SRM. Assuming purity of crude peptide is 80% for 10 mers and 70% for
15 mers, respectively.

| | | Average conc. | Conc. range | Ref_1 conc. | |
|---|---|---|---|---|---|
| Gene | | in MRM assay | in MRM | (pg/ml) | Ref_2 conc. |
| symbol | Peptide Sequence | (pg/ml) | assay (pg/ml) | [20, 21] | (pg/ml) [22] |
| A1BG | HQFLLTGDTQGR | 5.53E+07 | 1.23E+07 to | 2.20E+8 | 5.00E+07 |
| | | | 1.62E+08 | | |
| CFH | CTSTGWIPAPR | 5.51E+07 | 2.35E+07 to | | 5.70E+07 |
| | | | 1.07E+08 | | |
| IGFALS | VAGLLEDTFPGLLGLR | 6.52E+05 | 1.64E+05 to | | 1.50E+06 |
| | | | 1.36E+06 | | |
| PROC | TFVLNFIK | 1.00E+05 | 2.61E+04 to | 3.70E+06 | 6.50E+04 |
| | | | 1.72E+05 | | |
| RBP4 | YWGVASFLQK | 3.22E+06 | 1.72E+06 to | 3.17E+07 | 5.80E+08 |
| | | | 5.79E+06 | | |

A1BG, CFH and IGFALS Distinguish Controls from HCV Patients

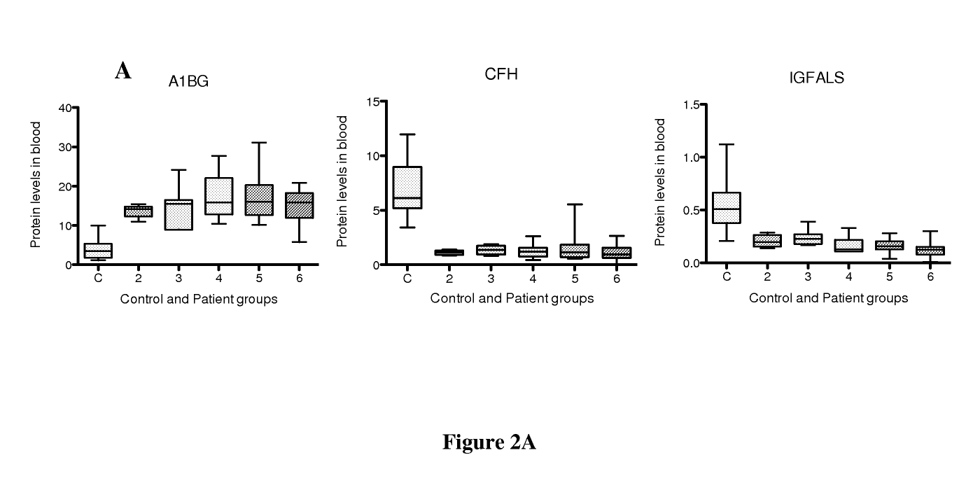

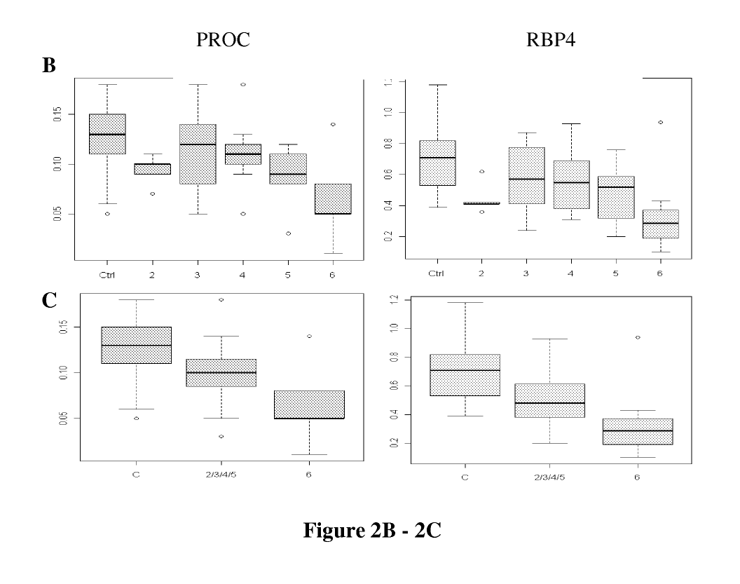

As shown in FIG. 2A, the average A1BG level in sera of all HCV patients in this study was significantly elevated compared with controls (P=7.4E-15), while CFH and IGFALS levels were significantly decreased in patients versus in controls (P=4.5E-08 and 5.7E-12, respectively). AUROC scores for controls vs. HALT-C patients were 0.99, 0.99, and 0.96 for A1BG, CFH, and IGFALS, respectively.

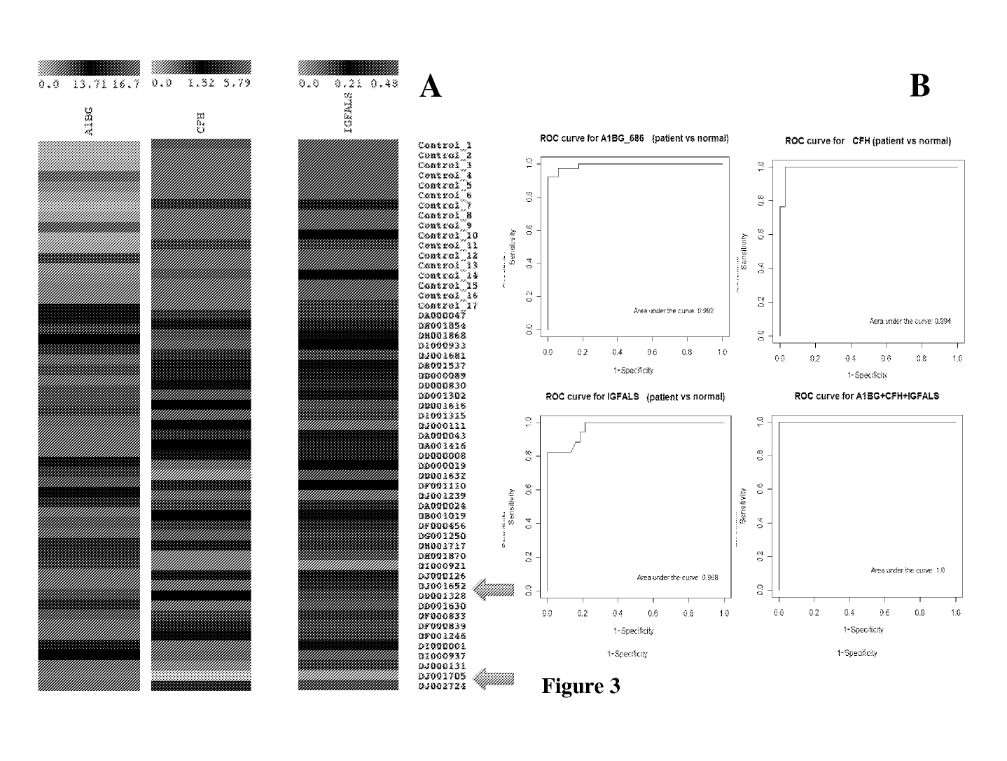

An integrated analysis of A1BG, CFH and IGFALS or A1BG with either of the other two markers resulted in a perfect AUROC score of 1.0 with predicted sensitivity and specificity of 100% for discriminating healthy controls and HCV-infected patients (FIGS. 3A and 3B). Sets of markers for discriminating acute and chronic liver diseases may be different, and the conventional liver markers AST and ALT may perform better in detecting acute liver injuries but are poor markers for chronic liver diseases. The 3-protein panel of A1BG, CFH, and IGFALS detects chronic liver diseases as shown. As further described below, two additional proteins, PROC and RBP4 may be used in conjunction with these three proteins individually or together to identify chronic liver conditions.

PROC and RBP4 Levels in Serum can Further Classify Patients with Fibrosis

The concentrations of two proteins—PROC and RBP4—showed good correlation with disease severity. Serum concentrations of PROC and RBP4 decreased as liver disease progressed. Box-and-whisker plots and Student's t-test showed that each protein can distinguish different disease stages (FIG. 2B).

With Student's t-test, the difference in serum concentrations of PROC or RBP4 between patients with earlier stages of fibrosis (Ishak 2-4) and patients with cirrhosis (Ishak 5-6) is significant. PROC appears to be a good marker to distinguish cirrhosis patients from those with fibrosis (p=0.004). RBP4 levels showed a similar decrease from normal control to Ishak 5-6 but the difference between Ishak 2-4 and 5-6 was not statistically significant—0.07 with outliers (0.02 without outliers). AUROC scores for PROC were 0.77 for controls vs. patients, 0.75 for Ishak <5 vs. Ishak≧5, and 0.83 for Ishak <5 vs. Ishak 6. AUROC scores for RBP4 were 0.79 for controls vs. patients, 0.68 for Ishak <5 vs. Ishak≧5, and 0.80 for Ishak <5 vs. Ishak 6.

Ishak 6 Patients are Effectively Distinguished by PROC and RBP4

When we combined Ishak 2-4 or 2-5 patients together and compared against the most advanced Ishak 6 patients, more impressive differences between these groups were revealed with an AUROC of 0.83 for PROC and 0.80 for RBP4.

The discrimination power of the 5 proteins in terms of AUROC is summarized as follows:

| | | AUROC | |
|---|---|---|---|
| Protein: | Analyzed groups | score | Comment |
| PROC: | Control vs. patient | 0.77 | Acceptable discrimination |
| PROC: | Ishak <5 vs. | 0.75 | Acceptable discrimination |
| | Ishak ≧5 | | |
| PROC: | Ishak <5 vs. Ishak 6 | 0.83 | Excellent discrimination |
| RBP4: | Control vs. patient | 0.79 | Acceptable discrimination |
| RBP4: | Ishak <5 vs. | 0.68 | Poor to fair discrimination |
| | Ishak ≧5 | | |
| RBP4: | Ishak <5 vs. Ishak 6 | 0.80 | Excellent discrimination |
| A1BG: | Control vs. patient | 0.99 | Perfect discrimination |
| CFH: | Control vs. patient | 0.99 | Perfect discrimination |
| IGFALS: | Control vs. patient | 0.96 | Perfect discrimination |

Multivariate Analysis

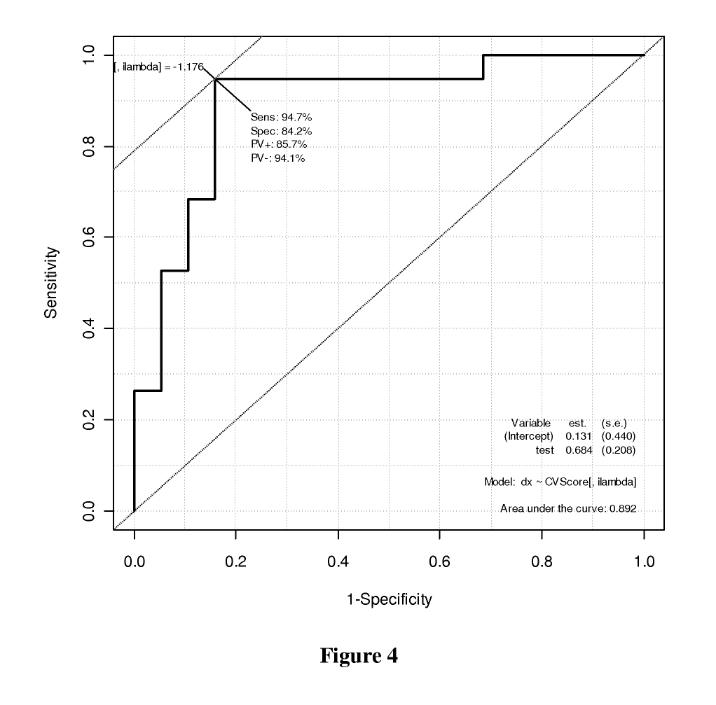

Multivariate analysis of Ishak 2-4 vs. 5-6 patients using PROC and RBP4 proteins, gender, and platelets gave a cross-validated AUROC=0.89. PROC, RBP4, and platelets distinguish advanced stages of fibrosis patients (Ishak 5-6) from patients in earlier stages with an impressive sensitivity of 95% and specificity of 84% (FIG. 4). A minor improvement (AUROC=0.72 vs. 0.69) was observed with platelets excluded in the analysis based on all available vs. only PROC and RBP4 proteins.

TABLE 1
Patient information on age, gender, platelet counts,
AST, ALT, and alkaline phosphatase levels in blood and
Ishak scores when enrolled in the HALT-C trial.
Table 1 HALT-C patient information

| | | | | | | Alkaline | Fi- |
|---|---|---|---|---|---|---|---|
| | | | Platelets | | | phos- | brosis- |
| Serum | | Gen- | (×1000/ | AST | ALT | phatase | Ishak |
| Sample ID | AGE | der | mm3) | (U/L) | (U/L) | (U/L) | score |
| DA 000047 | 48 | M | 232 | 89 | 121 | 78 | 2 |
| DI 000933 | 27 | M | 418 | 68 | 133 | 111 | 2 |
| DJ 001681 | 58 | M | 208 | 31 | 27 | 100 | 2 |
| DH 001854 | 48 | F | 222 | 44 | 67 | 86 | 2 |
| DH 001868 | 44 | F | 270 | 46 | 56 | 41 | 2 |
| DB 001537 | 47 | M | 132 | 46 | 73 | 80 | 3 |
| DD 000089 | 54 | M | 188 | 55 | 46 | 76 | 3 |
| DD 000830 | 53 | M | 243 | 69 | 113 | 85 | 3 |
| DD 001302 | 49 | M | 218 | 43 | 68 | 61 | 3 |
| DD 001616 | 45 | M | 216 | 35 | 60 | 71 | 3 |
| DJ 000111 | 51 | M | 208 | 64 | 75 | 97 | 3 |
| DI 001315 | 46 | F | 274 | 72 | 117 | 75 | 3 |
| DJ 000004 | 47 | F | 105 | 113 | 91 | 69 | 3 |
| DA 000043 | 51 | M | 280 | 58 | 82 | 46 | 4 |
| DD 000019 | 48 | M | 194 | 62 | 68 | 80 | 4 |
| DD 001632 | 49 | M | 135 | 89 | 141 | 82 | 4 |
| DF 001110 | 50 | M | 98 | 46 | 42 | 117 | 4 |
| DJ 001239 | 52 | M | 169 | 50 | 71 | 108 | 4 |
| DA 001416 | 56 | F | 143 | 98 | 121 | 61 | 4 |
| DD 000008 | 56 | F | 259 | 34 | 46 | 117 | 4 |
| DA 000024 | 53 | M | 132 | 157 | 261 | 68 | 5 |
| DA 000739 | 49 | M | 391 | 35 | 39 | 67 | 5 |
| DF 000456 | 51 | M | 114 | 128 | 160 | 83 | 5 |
| DG 001250 | 47 | M | 71 | 49 | 53 | 105 | 5 |
| DH 001717 | 59 | M | 106 | 41 | 68 | 133 | 5 |
| DI 000921 | 50 | M | 72 | 91 | 112 | 81 | 5 |
| DJ 000126 | 46 | M | 93 | 136 | 107 | 89 | 5 |
| DJ 001652 | 42 | M | 68 | 58 | 62 | 88 | 5 |
| DB 001019 | 56 | F | 225 | 33 | 25 | 80 | 5 |
| DH 001870 | 64 | F | 155 | 28 | 43 | 94 | 5 |
| DD 001328 | 67 | M | 140 | 28 | 42 | 128 | 6 |
| DD 001630 | 57 | M | 99 | 34 | 39 | 75 | 6 |
| DF 000833 | 60 | M | 98 | 100 | 159 | 117 | 6 |
| DF 001246 | 46 | M | 88 | 71 | 51 | 150 | 6 |
| DI 000001 | 56 | M | 93 | 45 | 60 | 76 | 6 |
| DJ 001705 | 56 | M | 106 | 89 | 78 | 142 | 6 |
| DJ 002724 | 66 | M | 167 | 231 | 137 | 301 | 6 |
| DF 000839 | 50 | F | 176 | 244 | 291 | 127 | 6 |
| DI 000937 | 50 | F | 83 | 213 | 212 | 307 | 6 |
| DJ 000131 | 46 | F | 95 | 95 | 77 | 174 | 6 |

TABLE 2
87 proteins previously detected by tandem mass spectrometry and tested by SRM with light peptides

| | | | | | | | | | |
|---|---|---|---|---|---|---|---|---|---|
| A1BG** | AGXT* | AHSG** | ALDOB | ALT1# | ALT2# | AMBP** | ANG | APCS** | APOA5 |
| APOC1 | APOC2** | APOC4* | APOF | APOH** | ASGR2 | ASL | AST1**# | AST2# | BHMT |
| C8A | C8B* | C8G** | C9** | CES1* | CFB** | CFH** | CFHR2** | CFHR4 | CHI3L1# |
| CP** | CPB2** | CPS1* | DPYS | F2**# | F9** | F10* | F12** | FAH** | FBP1* |
| FCN2 | FETUB** | FGA* | FGB* | FGG* | GC** | GNMT | GOLM1# | GRN# | HABP2** |
| HAMP | HAO1 | HGD | HGFAC** | HMGCS2* | HPD | HPR* | HPX | HRG*# | HSPA9# |
| IDH1 | IGFALS** | ITIH1** | ITIH2** | ITIH3** | ITIH4* | LBP** | LCAT** | LPA** | MASP2 |
| MAT1A* | MBL2 | MDH | MMP9# | PEX1 | PGLYRP2** | PKLR* | PROC** | RBP4** | SAA4 |
| SERPINF2** | SPP2 | TAT | TIMP1* | TNC**# | UPB1 | VTN** | | | |

*endogenous peptide detected in pooled control plasma
**endogenous peptide detected in control sera
#proteins used in clinic practice or previously reported as liver biomarker candidates

TABLE 3
SRM monitoring conditions of 38 proteins that detectable in control sera. Many
proteins are represented by two or more peptides.
K: ¹³C6¹⁵N2 isotopic-labeled lysine, R: ¹³C6¹⁵N4 isotopic-labeled arginine

| | | Peptide | MH+ | Z | Transitions | | | | |
|---|---|---|---|---|---|---|---|---|---|
| Symbol | Description | Sequence | (mono) | (Q1) | Q1 | Q3 | | | |
| A1BG | Alpha-1-B | HQFLLTGDTQGR | 1372.7 | 2 | 686.9 | 734.3 | 847.4 | 960.5 | 1107.6 |
| | glycoprotein | HQFLLTGDTQGR | 1382.7 | | 691.9 | 744.3 | 857.4 | 970.5 | 1117.6 |
| A1BG | Alpha-1-B | HQFLLTGDTQGR | 1372.7 | 3 | 458.2 | 367.7 | 576.3 | 633.3 | 734.3 |
| | glycoprotein | HQFLLTGDTQGR | 1382.7 | | 461.6 | 372.7 | 586.3 | 643.3 | 744.3 |
| A1BG | Alpha-1-B | SGLSTGWTQLSK | 1264.7 | 2 | 632.8 | 762.4 | 819.4 | 920.5 | 1007.5 |
| | glycoprotein | SGLSTGWTQLSK | 1272.7 | | 636.8 | 770.4 | 827.4 | 928.5 | 1015. |
| AHSG | Alpha-2-HS- | EHAVEGDCDFQLLK | 1660.7 | 2 | 830.9 | 1095.5 | 1224.6 | 1323.6 | 1394.7 |
| | glycoprotein | EHAVEGDCDFQLLK | 1668.7 | | 834.9 | 1103.5 | 1232.6 | 1331.6 | 1402.7 |
| AHSG | Alpha-2-HS- | HTLNQIDEVK | 1196.6 | 2 | 598.8 | 731.4 | 845.4 | 958.5 | 1059.6 |
| | glycoprotein | HTLNQIDEVK | 1204.6 | | 602.8 | 739.4 | 853.4 | 966.5 | 1067.6 |
| AMBP | Alpha-1- | AFIQLWAFDAVK | 1408.8 | 2 | 704.9 | 579.3 | 650.4 | 836.4 | 949.5 |
| | microglobulin/ | AFIQLWAFDAVK | 1416.8 | | 708.9 | 587.3 | 658.4 | 844.4 | 957.5 |
| | bikunin | | | | | | | | |
| AMBP | Alpha-1- | GVCEETSGAYEK | 1329.5 | 2 | 665.3 | 755.4 | 884.4 | 1013.4 | 1173.5 |
| | microglobulin/ | GVCEETSGAYEK | 1337.5 | | 669.3 | 763.4 | 892.4 | 1021.4 | 1181.5 |
| | bikunin | | | | | | | | |
| APCS | Amyloid P | AYSLFSYNTQGR | 1406.7 | 2 | 703.84 | 575.3 | 738.4 | 825.4 | 972.5 |
| | component, | AYSLFSYNTQGR | 1416.7 | | 708.84 | 585.3 | 748.4 | 835.4 | 982.5 |
| | serum | | | | | | | | |
| APCS | Amyloid P | VGEYSLYIGR | 1156.6 | 2 | 578.8 | 508.3 | 621.4 | 708.4 | 871.5 |
| | component, | VGEYSLYIGR | 1166.6 | | 583.8 | 518.3 | 631.4 | 718.4 | 881.5 |
| | serum | | | | | | | | |
| APOC2 | Apolipoprotein | ESLSSYWESAK | 1286.6 | 2 | 643.8 | 783.4 | 870.4 | 957.4 | 1070.5 |
| | C-II | ESLSSYWESAK | 1294.6 | | 647.8 | 791.4 | 878.4 | 965.4 | 1078.5 |
| APOC2 | Apolipoprotein | TAAQNLYEK | 1037.5 | 2 | 519.3 | 552.3 | 666.4 | 794.4 | 865.4 |
| | C-II | TAAQNLYEK | 1045.5 | | 523.3 | 560.3 | 674.4 | 802.4 | 873.4 |
| APOH | Apolipoprotein | ATVVYQGER | 1037.5 | 2 | 511.8 | 489.2 | 652.3 | 751.4 | 850.4 |
| | H | ATVVYQGER | 1045.5 | | 516.8 | 499.2 | 662.3 | 761.4 | 860.4 |
| APOH | Apolipoprotein | EHSSLAFWK | 1104.6 | 2 | 552.8 | 551.3 | 664.4 | 751.4 | 838.5 |
| | H | EHSSLAFWK | 1112.6 | | 556.8 | 559.3 | 672.4 | 759.4 | 846.5 |
| AST1 | glutamic- | LALGDDSPALK | 1099.6 | 2 | 550.3 | 745.4 | 802.4 | 915.5 | 986.5 |
| | oxaloacetic | LALGDDSPALK | 1107.6 | | 554.3 | 753.4 | 810.4 | 923.5 | 994.5 |
| | transaminase 1 | | | | | | | | |
| AST1 | glutamic- | TDDCHPWVLPVVK | 1565.8 | 3 | 522.6 | 617.8 | 654.5 | 675.4 | 732.9 |
| | oxaloacetic | TDDCHPWVLPVVK | 1573.8 | | 525.3 | 621.8 | 662.5 | 679.4 | 736.9 |
| | transaminase 1 | | | | | | | | |
| C8G | Complement | SLPVSDSVLSGFEQR | 1620.8 | 3 | 541.0 | 579.3 | 636.3 | 662. | 710.9 |
| | component 8, | SLPVSDSVLSGFEQR | 1630.8 | | 544.3 | 589.3 | 646.3 | 667.3 | 715.9 |
| | gamma | | | | | | | | |
| | polypeptide | | | | | | | | |
| C9 | Complement | AIEDYINEFSVR | 1455.7 | 2 | 728.4 | 637.3 | 751.4 | 864.5 | 1271.6 |
| | component 9 | AIEDYINEFSVR | 1465.7 | | 733.4 | 647.3 | 761.4 | 874.5 | 1281.6 |
| CFB | Complement | DFHINLFQVLPWLK | 1770.0 | 3 | 590.7 | 543.3 | 656.4 | 755.5 | 883.5 |
| | factor B | DFHINLFQVLPWLK | 1778.0 | | 593.3 | 551.3 | 664.4 | 763.5 | 891.5 |
| CFB | Complement | YGLVTYATYPK | 1275.7 | 2 | 638.3 | 579.3 | 742.9 | 843.4 | 942.5 |
| | factor B | YGLVTYATYPK | 1283.7 | | 642.3 | 587.3 | 750.9 | 851.4 | 950.5 |
| CFH | Complement | CTSTGWIPAPR | 1245.6 | 2 | 623.3 | 440.3 | 553.4 | 739.4 | 796.5 |
| | factor H | CTSTGWIPAPR | 1255.6 | | 628.3 | 450.3 | 563.4 | 749.4 | 806.5 |
| CFHR2 | Complement | ITCAEEGWSPTPK | 1475.7 | 2 | 738.4 | 529.3 | 772.4 | 901.4 | 1030.5 |
| | factor H- | ITCAEEGWSPTPK | 1483.7 | | 742.4 | 537.3 | 780.4 | 909.4 | 1038.5 |
| | related 2 | | | | | | | | |
| CFHR2 | Complement | TGDIVEFVCK | 1167.6 | 2 | 584.3 | 553.3 | 682.3 | 781.4 | 894.5 |
| | factor H- | TGDIVEFVCK | 1175.6 | | 588.3 | 561.3 | 690.3 | 789.4 | 902.5 |
| | related 2 | | | | | | | | |
| CP | Ceruloplasmin | ALYLQYTDETFR | 1519.7 | 2 | 760.4 | 667.3 | 931.4 | 1059.5 | 1172.6 |
| | (ferroxidase) | ALYLQYTDETFR | 1529.7 | | 765.4 | 677.3 | 941.4 | 1069.5 | 1182.6 |
| CP | Ceruloplasmin | IYHSHIDAPK | 1180.6 | 3 | 394.2 | 430.2 | 452.7 | 534.3 | 767.4 |
| | (ferroxidase) | IYHSHIDAPK | 1188.6 | | 396.9 | 438.2 | 456.7 | 538.3 | 775.4 |
| CPB2 | Carboxy- | SFYANNHCIGTDLNR | 1781.8 | 2 | 891.4 | 675.3 | 788.4 | 948.5 | 1085.5 |
| | peptidase | SFYANNHCIGTDLNR | 1791.8 | | 896.4 | 685.3 | 798.4 | 958.5 | 1095.5 |
| | B2 (plasma) | | | | | | | | |
| CPB2 | Carboxy- | SFYANNHCIGTDLNR | 1781.8 | 3 | 594.6 | 657. | 692.8 | 774.4 | 948.5 |
| | peptidase | SFYANNHCIGTDLNR | 1791.8 | | 597.9 | 662.3 | 697.8 | 779.4 | 958.5 |
| | B2 (plasma) | | | | | | | | |
| F12 | Coagulation | CFEPQLLR | 1062.5 | 2 | 531.8 | 401.3 | 626.4 | 755.4 | 902.5 |
| | factor XII | CFEPQLLR | 1072.5 | | 536.8 | 411.3 | 636.4 | 765.4 | 912.5 |
| F2 | Coagulation | ELLESYIDGR | 1194.6 | 2 | 597.8 | 460.3 | 623.3 | 710.4 | 839.4 |
| | factor II | ELLESYIDGR | 1204.6 | | 602.8 | 470.3 | 633.3 | 720.4 | 849.4 |
| | (thrombin) | | | | | | | | |
| F2 | Coagulation | TATSEYQTFFNPR | 1561.7 | 2 | 781.4 | 533.3 | 680.6 | 909.5 | 1072.5 |
| | factor II | TATSEYQTFFNPR | 1571.7 | | 786.4 | 543.3 | 690.6 | 919.5 | 1082. |
| | (thrombin) | | | | | | | | |
| F9 | Coagulation | WIVTAAHCVETGVK | 1570.8 | 3 | 524.3 | 536.3 | 586.8 | 636.3 | 692.9 |
| | factor IX | WIVTAAHCVETGVK | 1578.8 | | 526.9 | 540.3 | 590.8 | 640.3 | 696.9 |
| FAH | Fumarylaceto- | VFLQNLLSVSQAR | 1474.8 | 2 | 737.9 | 560.3 | 647.4 | 760.4 | 987.6 |
| | acetate | VFLQNLLSVSQAR | 1484.8 | | 742.9 | 570.3 | 657.4 | 770.4 | 997.6 |
| | hydrolase | | | | | | | | |
| FETUB | Fetuin B | IFFESVYGQCK | 1377.63 | 2 | 689.3 | 492.2 | 655.3 | 841.4 | 1117.5 |
| | | IFFESVYGQCK | 1385.63 | | 693.3 | 500.2 | 663.3 | 849.4 | 1125.5 |
| GC | Group-specific | HLSLLTTLSNR | 1254.7 | 3 | 418.9 | 376.2 | 489.3 | 590.3 | 691.4 |
| | component | HLSLLTTLSNR | 1264.7 | | 422.3 | 386.2 | 499.3 | 600.3 | 701.4 |
| | (vitamin D | | | | | | | | |
| | binding | | | | | | | | |
| | protein) | | | | | | | | |
| GC | Group-specific | VCSQYAAYGEK | 1275.6 | 2 | 638.3 | 496.2 | 567.3 | 801.4 | 1016.5 |
| | component | VCSQYAAYGEK | 1283.6 | | 642.3 | 504.2 | 575.3 | 809.4 | 1024.5 |
| | (vitamin D | | | | | | | | |
| | binding | | | | | | | | |
| | protein) | | | | | | | | |
| HABP2 | Hyaluronan | DEIPHNDIALLK | 1377.7 | 3 | 459.9 | 444.3 | 462.3 | 510.8 | 567.3 |
| | binding | DEIPHNDIALLK | 1385.7 | | 462.6 | 452.3 | 466.3 | 514.8 | 571.3 |
| | protein 2 | | | | | | | | |
| HABP2 | Hyaluronan | GQCLITQSPPYYR | 1582.7 | 2 | 791.9 | 695.4 | 782.4 | 910.4 | 1011.5 |
| | binding | GQCLITQSPPYYR | 1592.7 | | 796.9 | 705.4 | 792.4 | 920.4 | 1021.5 |
| | protein 2 | | | | | | | | |
| HGFAC | HGF | TTDVTQTFGIEK | 1339.7 | 2 | 670.3 | 694.4 | 822.4 | 923.5 | 1137.6 |
| | activator | TTDVTQTFGIEK | 1347.7 | | 674.3 | 702.4 | 830.4 | 931.5 | 1145.6 |
| HGFAC | HGF | VANYVDWINDR | 1364.7 | 2 | 682.8 | 703.4 | 818.4 | 917.5 | 1080.5 |
| | activator | VANYVDWINDR | 1374.7 | | 687.8 | 713.4 | 828.4 | 927.5 | 1090.5 |
| HP | Haptoglobin | VGYVSGWGR | 980.5 | 3 | 490.8 | 475.2 | 562.3 | 661.3 | 824.4 |
| | | VGYVSGWGR | 990.5 | | 495.8 | 485.2 | 572.3 | 671.3 | 834.4 |
| HP | Haptoglobin | VTSIQDWVQK | 1203.6 | 2 | 602.3 | 560.3 | 675.4 | 803.4 | 1003.5 |
| | | VTSIQDWVQK | 1211.6 | | 606.3 | 568.3 | 683.4 | 811.4 | 1011.5 |
| HRG | Histidine | DSPVLIDFFEDTER | 1682.8 | 2 | 841.9 | 796.4 | 943.4 | 1058.4 | 1171.5 |
| | rich | DSPVLIDFFEDTER | 1692.8 | | 846.9 | 806.4 | 953.4 | 1068.4 | 1181.5 |
| | glycoprotein | | | | | | | | |
| HRG | Histidine | GGEGTGYFVDFSVR | 1490.7 | 2 | 745.9 | 869.6 | 1032.5 | 1089.5 | 1190.6 |
| | rich | GGEGTGYFVDFSVR | 1500.7 | | 750.9 | 879.6 | 1042.5 | 1099.5 | 1200.6 |
| | glycoprotein | | | | | | | | |
| IGFALS | Insulin-like | LHSLHLEGSCLGR | 1478.7 | 3 | 493.6 | 514.8 | 614.8 | 649.3 | 778.4 |
| | growth | LHSLHLEGSCLGR | 1488.7 | | 496.9 | 519.8 | 619.8 | 659.3 | 788.4 |
| | factor | | | | | | | | |
| | binding | | | | | | | | |
| | protein, | | | | | | | | |
| | acid labile | | | | | | | | |
| | subunit | | | | | | | | |
| IGFALS | Insulin-like | VAGLLEDTFPGLLGLR | 1671.0 | 2 | 836.0 | 725.5 | 872.5 | 1088.6 | 1217.7 |
| | growth | VAGLLEDTFPGLLGLR | 1681.0 | | 841.0 | 735.5 | 882.5 | 1098.6 | 1227.7 |
| | factor | | | | | | | | |
| | binding | | | | | | | | |
| | protein, | | | | | | | | |
| | acid labile | | | | | | | | |
| | subunit | | | | | | | | |
| ITIH1 | Inter-alpha | AAISGENAGLVR | 1157.6 | 2 | 579.3 | 629.4 | 758.4 | 815.4 | 902.5 |
| | (globulin) | AAISGENAGLVR | 1167.6 | | 584. | 639.4 | 768.4 | 825.4 | 912.5 |
| | inhibitor | | | | | | | | |
| | H1 | | | | | | | | |
| ITIH1 | Inter-alpha | LDAQASFLPK | 1089.6 | 2 | 545.3 | 591.4 | 662.4 | 790.5 | 861.5 |
| | (globulin) | LDAQASFLPK | 1097.6 | | 549.3 | 599.4 | 670.4 | 798.5 | 869.5 |
| | inhibitor | | | | | | | | |
| | H1 | | | | | | | | |
| ITIH2 | Inter-alpha | TEVNVLPGAK | 1027.6 | 2 | 514.3 | 372.2 | 485.3 | 698.4 | 797.5 |
| | (globulin) | TEVNVLPGAK | 1035.6 | | 518.3 | 380.2 | 493.3 | 706.4 | 805.5 |
| | inhibitor | | | | | | | | |
| | H2 | | | | | | | | |
| ITIH3 | Inter-alpha | DYIFGNYIER | 1289.6 | 2 | 645.3 | 694.4 | 751.4 | 898.4 | 1011.5 |
| | (globulin) | DYIFGNYIER | 1299.6 | | 650.3 | 704.4 | 761.4 | 908.4 | 1021.5 |
| | inhibitor | | | | | | | | |
| | H3 | | | | | | | | |
| LBP | Lipopoly- | ITLPDFTGDLR | 1247.7 | 2 | 624.3 | 708.4 | 823.4 | 920.5 | 1033.5 |
| | saccharide | ITLPDFTGDLR | 1257.7 | | 629.3 | 718.4 | 833.4 | 930.5 | 1043.5 |
| | binding | | | | | | | | |
| | protein | | | | | | | | |
| LCAT | Lecithin- | SSGLVSNAPGVQIR | 1384.8 | 2 | 692.9 | 740. | 854.5 | 941.5 | 1040.6 |
| | cholesterol | SSGLVSNAPGVQIR | 1394.8 | | 697.9 | 750.45 | 864.5 | 951.5 | 1050.6 |
| | acyltrans- | | | | | | | | |
| | ferase | | | | | | | | |
| LCAT | Lecithin- | TYSVEYLDSSK | 1291.6 | 2 | 646.3 | 712.4 | 841.39 | 940.5 | 1027.5 |
| | cholesterol | TYSVEYLDSSK | 1299.6 | | 650.3 | 720.4 | 849.41 | 948.5 | 1035.5 |
| | acyltrans- | | | | | | | | |
| | ferase | | | | | | | | |
| LPA | Lipoprotein, | NPDAVAAPYCYTR | 1497.7 | 2 | 749.3 | 859.4 | 930.41 | 1001.5 | 1100.5 |
| | Lp(a) | NPDAVAAPYCYTR | 1507.7 | | 754.3 | 869.4 | 940.42 | 1011.5 | 1110.5 |
| PGLYRP2 | Peptidoglycan | PSLSHLLSQYYGAGVAR | 1819.0 | 3 | 607.0 | 473.3 | 530.30 | 693.4 | 856.4 |
| | recognition | PSLSHLLSQYYGAGVAR | 1829.0 | | 610.3 | 483.3 | 540.31 | 703.4 | 866.4 |
| | protein 2 | | | | | | | | |
| PGLYRP2 | Peptidoglycan | TDCPGDALFDLLR | 1492.7 | 2 | 746.9 | 663.4 | 776.47 | 847.5 | 1116.6 |
| | recognition | TDCPGDALFDLLR | 1502.7 | | 751. | 673.4 | 786.47 | 857.5 | 1126.6 |
| | protein 2 | | | | | | | | |
| PON1 | Paraoxonase | EVQPVELPNCNLVK | 1638.8 | 2 | 819.9 | 844.4 | 957.5 | 1086.6 | 1282.7 |
| | 1 | EVQPVELPNCNLVK | 1646.8 | | 823.9 | 852.4 | 965.5 | 1094.6 | 1290.7 |
| PON1 | Paraoxonase | IFFYDSENPPASEVLR | 1883.9 | 2 | 942.5 | 603.4 | 868.5 | 982.5 | 1198.6 |
| | 1 | IFFYDSENPPASEVLR | 1893.9 | | 947.5 | 613.4 | 878.5 | 992.5 | 1208.6 |
| PON1 | Paraoxonase | IQNILTEEPK | 1184.7 | 2 | 592.8 | 603.3 | 716.4 | 829.5 | 943.5 |
| | 1 | IQNILTEEPK | 1192.7 | | 596.8 | 611.3 | 724.4 | 837.5 | 951.5 |
| PROC | Protein C | EVFVHPNYSK | 1219.6 | 2 | 610.3 | 608.3 | 745.4 | 844.4 | 991.5 |
| | | EVFVHPNYSK | 1227.6 | | 614.3 | 616.3 | 753.4 | 852.4 | 999.5 |
| PROC | Protein C | TFVLNFIK | 981.6 | 2 | 491.3 | 521.3 | 634.4 | 733.5 | 880.5 |
| | | TFVLNFIK | 989.6 | | 495.3 | 529.3 | 642.4 | 741.5 | 888.5 |
| RBP4 | Retinol | YWGVASFLQK | 1198.6 | 2 | 599.8 | 535.3 | 622.4 | 693.4 | 849.5 |
| | binding | YWGVASFLQK | 1206.6 | | 603.8 | 543.3 | 630.4 | 701.4 | 857.5 |
| | protein 4, | | | | | | | | |
| | plasma | | | | | | | | |
| SERPINF2 | Serpin | LCQDLGPGAFR | 1233.6 | 2 | 617.3 | 547.3 | 604.3 | 717.4 | 960.5 |
| | peptidase | LCQDLGPGAFR | 1243.6 | | 622.3 | 557.3 | 614.3 | 727.4 | 970.5 |
| | inhibitor, | | | | | | | | |
| | clade F, | | | | | | | | |
| | member 2 | | | | | | | | |
| TNC | Tenascin C | EEFWLGLDNLNK | 1477.7 | 2 | 739.4 | 773.4 | 886.5 | 1072.6 | 1219.7 |
| | (hexabrachion) | EEFWLGLDNLNK | 1485.7 | | 743.4 | 781.4 | 894.5 | 1080.6 | 1227.7 |
| TNC | Tenascin C | ETFTTGLDAPR | 1207.6 | 2 | 604.3 | 628.3 | 729.4 | 830.4 | 977.5 |
| | (hexabrachion) | ETFTTGLDAPR | 1217.6 | | 609.3 | 638.3 | 739.4 | 840.4 | 987.5 |
| VTN | Vitronectin | DVWGIEGPIDAAFTR | 1646.8 | 2 | 823.9 | 890.5 | 947.5 | 1076.5 | 1246.6 |
| | | DVWGIEGPIDAAFTR | 1656.8 | | 828.9 | 900.5 | 957.5 | 1086.5 | 1256.6 |
| VTN | Vitronectin | GQYCYELDEK | 1304.5 | 2 | 652.8 | 504.3 | 633.3 | 796.4 | 956.4 |
| | | GQYCYELDEK | 1312.5 | | 656.8 | 512.3 | 641.3 | 804.4 | 964.4 |

1. A method to distinguish a subject with chronic or acute abnormal liver condition from a subject without said liver condition which method comprises:
assessing the level of alpha-1-B glycoprotein (A1BG) or complement Factor H (CFH) or insulin-like growth factor binding protein acid labeled subunit (IGFALS) or combinations thereof in the blood or fraction thereof of said subject
whereby an increased level of A1BG or a decreased level of CFH or IGFALS or a combination thereof in the blood or fraction thereof of said subject as compared to normal individuals identifies said subject as having a chronic or acute abnormal liver condition.

2. The method of claim 1 which further includes
assessing the level of protein C (PROC) and/or retinal binding protein 4 (RBP4) in the blood or a fraction thereof of said subject
wherein decreased levels of one or both of said PROC and RBP4 as compared to normal individuals further supports identifying the subject as having a chronic or acute abnormal liver condition.

3. The method of claim 1 wherein the condition is due to hepatitis infection.

4. The method of claim 2 wherein the condition is due to hepatitis infection.

5. The method of claim 3 wherein the hepatitis is Hepatitis C.

6. The method of claim 4 wherein the hepatitis is Hepatitis C.

7. A method to determine severity of liver fibrosis in a subject exhibiting an abnormal liver condition which method comprises
assessing the level of protein C (PROC) and/or retinal binding protein 4 (RBP4) in the blood or a fraction thereof of said subject
wherein decreased levels of one or both of said PROC and RBP4 as compared to normal individuals correlates positively with the severity of fibrosis of the liver in said subject.

8. The method of claim 7 wherein the condition is due to hepatitis infection.

9. The method of claim 8 wherein the hepatitis is Hepatitis C.

10. A panel of reagents comprising detection reagents for one or more of A1GB, CFH and IGFALS.

11. The panel of claim 10 which further includes detection reagents for PROC and/or RBP4.

12. The panel of claim 10 wherein the reagents are antibodies or aptamers.

13. The panel of claim 11 wherein the reagents are antibodies or aptamers.

14. A panel of reagents comprising detection reagents for one or more of PROC and RBP4.

15. The panel of claim 14 wherein the reagents are antibodies or aptamers.

16. Antibodies or aptamers that specifically bind any one of A1BG, CFH, IGFALS, PROC and RBP4.


SEQUENCE LISTING (63 sequences)

SEQ ID NO: 1 - protein, 12 aa, Artificial Sequence
  synthetically constructed peptide
HQFLLTGDTQGR

SEQ ID NO: 2 - protein, 12 aa, Artificial Sequence
  synthetically constructed peptide
SGLSTGWTQLSK

SEQ ID NO: 3 - protein, 14 aa, Artificial Sequence
  synthetically constructed peptide
EHAVEGDCDFQLLK

SEQ ID NO: 4 - protein, 10 aa, Artificial Sequence
  synthetically constructed peptide
HTLNQIDEVK

SEQ ID NO: 5 - protein, 12 aa, Artificial Sequence
  synthetically constructed peptide
AFIQLWAFDAVK

SEQ ID NO: 6 - protein, 12 aa, Artificial Sequence
  synthetically constructed peptide
GVCEETSGAYEK

SEQ ID NO: 7 - protein, 12 aa, Artificial Sequence
  synthetically constructed peptide
AYSLFSYNTQGR

SEQ ID NO: 8 - protein, 10 aa, Artificial Sequence
  synthetically constructed peptide
VGEYSLYIGR

SEQ ID NO: 9 - protein, 11 aa, Artificial Sequence
  synthetically constructed peptide
ESLSSYWESAK

SEQ ID NO: 10 - protein, 9 aa, Artificial Sequence
  synthetically constructed peptide
TAAQNLYEK

SEQ ID NO: 11 - protein, 9 aa, Artificial Sequence
  synthetically constructed peptide
ATVVYQGER

SEQ ID NO: 12 - protein, 9 aa, Artificial Sequence
  synthetically constructed peptide
EHSSLAFWK

SEQ ID NO: 13 - protein, 11 aa, Artificial Sequence
  synthetically constructed peptide
LALGDDSPALK

SEQ ID NO: 14 - protein, 13 aa, Artificial Sequence
  synthetically constructed peptide
TDDCHPWVLPVVK

SEQ ID NO: 15 - protein, 15 aa, Artificial Sequence
  synthetically constructed peptide
SLPVSDSVLSGFEQR

SEQ ID NO: 16 - protein, 12 aa, Artificial Sequence
  synthetically constructed peptide
AIEDYINEFSVR

SEQ ID NO: 17 - protein, 14 aa, Artificial Sequence
  synthetically constructed peptide
DFHINLFQVLPWLK

SEQ ID NO: 18 - protein, 11 aa, Artificial Sequence
  synthetically constructed peptide
YGLVTYATYPK

SEQ ID NO: 19 - protein, 11 aa, Artificial Sequence
  synthetically constructed peptide
CTSTGWIPAPR

SEQ ID NO: 20 - protein, 13 aa, Artificial Sequence
  synthetically constructed peptide
ITCAEEGWSPTPK

SEQ ID NO: 21 - protein, 10 aa, Artificial Sequence
  synthetically constructed peptide
TGDIVEFVCK

SEQ ID NO: 22 - protein, 12 aa, Artificial Sequence
  synthetically constructed peptide
ALYLQYTDETFR

SEQ ID NO: 23 - protein, 10 aa, Artificial Sequence
  synthetically constructed peptide
IYHSHIDAPK

SEQ ID NO: 24 - protein, 15 aa, Artificial Sequence
  synthetically constructed peptide
SFYANNHCIGTDLNR

SEQ ID NO: 25 - protein, 8 aa, Artificial Sequence
  synthetically constructed peptide
CFEPQLLR

SEQ ID NO: 26 - protein, 10 aa, Artificial Sequence
  synthetically constructed peptide
ELLESYIDGR

SEQ ID NO: 27 - protein, 13 aa, Artificial Sequence
  synthetically constructed peptide
TATSEYQTFFNPR

SEQ ID NO: 28 - protein, 14 aa, Artificial Sequence
  synthetically constructed peptide
WIVTAAHCVETGVK

SEQ ID NO: 29 - protein, 13 aa, Artificial Sequence
  synthetically constructed peptide
VFLQNLLSVSQAR

SEQ ID NO: 30 - protein, 11 aa, Artificial Sequence
  synthetically constructed peptide
IFFESVYGQCK

SEQ ID NO: 31 - protein, 11 aa, Artificial Sequence
  synthetically constructed peptide
HLSLLTTLSNR

SEQ ID NO: 32 - protein, 11 aa, Artificial Sequence
  synthetically constructed peptide
VCSQYAAYGEK

SEQ ID NO: 33 - protein, 12 aa, Artificial Sequence
  synthetically constructed peptide
DEIPHNDIALLK

SEQ ID NO: 34 - protein, 13 aa, Artificial Sequence
  synthetically constructed peptide
GQCLITQSPPYYR

SEQ ID NO: 35 - protein, 12 aa, Artificial Sequence
  synthetically constructed peptide
TTDVTQTFGIEK

SEQ ID NO: 36 - protein, 11 aa, Artificial Sequence
  synthetically constructed peptide
VANYVDWINDR

SEQ ID NO: 37 - protein, 9 aa, Artificial Sequence
  synthetically constructed peptide
VGYVSGWGR

SEQ ID NO: 38 - protein, 10 aa, Artificial Sequence
  synthetically constructed peptide
VTSIQDWVQK

SEQ ID NO: 39 - protein, 14 aa, Artificial Sequence
  synthetically constructed peptide
DSPVLIDFFEDTER

SEQ ID NO: 40 - protein, 14 aa, Artificial Sequence
  synthetically constructed peptide
GGEGTGYFVDFSVR

SEQ ID NO: 41 - protein, 13 aa, Artificial Sequence
  synthetically constructed peptide
LHSLHLEGSCLGR

SEQ ID NO: 42 - protein, 16 aa, Artificial Sequence
  synthetically constructed peptide
VAGLLEDTFPGLLGLR

SEQ ID NO: 43 - protein, 12 aa, Artificial Sequence
  synthetically constructed peptide
AAISGENAGLVR

SEQ ID NO: 44 - protein, 10 aa, Artificial Sequence
  synthetically constructed peptide
LDAQASFLPK

SEQ ID NO: 45 - protein, 10 aa, Artificial Sequence
  synthetically constructed peptide
TEVNVLPGAK

SEQ ID NO: 46 - protein, 10 aa, Artificial Sequence
  synthetically constructed peptide
DYIFGNYIER

SEQ ID NO: 47 - protein, 11 aa, Artificial Sequence
  synthetically constructed peptide
ITLPDFTGDLR

SEQ ID NO: 48 - protein, 14 aa, Artificial Sequence
  synthetically constructed peptide
SSGLVSNAPGVQIR

SEQ ID NO: 49 - protein, 11 aa, Artificial Sequence
  synthetically constructed peptide
TYSVEYLDSSK

SEQ ID NO: 50 - protein, 13 aa, Artificial Sequence
  synthetically constructed peptide
NPDAVAAPYCYTR

SEQ ID NO: 51 - protein, 17 aa, Artificial Sequence
  synthetically constructed peptide
PSLSHLLSQYYGAGVAR

SEQ ID NO: 52 - protein, 13 aa, Artificial Sequence
  synthetically constructed peptide
TDCPGDALFDLLR

SEQ ID NO: 53 - protein, 14 aa, Artificial Sequence
  synthetically constructed peptide
EVQPVELPNCNLVK

SEQ ID NO: 54 - protein, 16 aa, Artificial Sequence
  synthetically constructed peptide
IFFYDSENPPASEVLR

SEQ ID NO: 55 - protein, 10 aa, Artificial Sequence
  synthetically constructed peptide
IQNILTEEPK

SEQ ID NO: 56 - protein, 10 aa, Artificial Sequence
  synthetically constructed peptide
EVFVHPNYSK

SEQ ID NO: 57 - protein, 8 aa, Artificial Sequence
  synthetically constructed peptide
TFVLNFIK

SEQ ID NO: 58 - protein, 10 aa, Artificial Sequence
  synthetically constructed peptide
YWGVASFLQK

SEQ ID NO: 59 - protein, 11 aa, Artificial Sequence
  synthetically constructed peptide
LCQDLGPGAFR

SEQ ID NO: 60 - protein, 12 aa, Artificial Sequence
  synthetically constructed peptide
EEFWLGLDNLNK

SEQ ID NO: 61 - protein, 11 aa, Artificial Sequence
  synthetically constructed peptide
ETFTTGLDAPR

SEQ ID NO: 62 - protein, 15 aa, Artificial Sequence
  synthetically constructed peptide
DVWGIEGPIDAAFTR

SEQ ID NO: 63 - protein, 10 aa, Artificial Sequence
  synthetically constructed peptide
GQYCYELDEK